(19) United States
(12) Patent Application Publication
(10) Pub. No.: US 2026/0308096 A1
(43) Pub. Date: Oct. 8, 2026

(54) COMPOUND, COMPOSITION, AND PACKAGE STRUCTURE

(71) Applicant: INDUSTRIAL TECHNOLOGY RESEARCH INSTITUTE, Hsinchu (TW)

(72) Inventors: Meei-Yu HSU, Hsinchu City (TW); Chih-Hao LIN, Taoyuan City (TW); Yu-Ling FAN, New Taipei City (TW); Xiao-Wei WU, Taoyuan City (TW); Kai-Chi CHEN, Zhubei City (TW)

(73) Assignee: INDUSTRIAL TECHNOLOGY RESEARCH INSTITUTE, Hsinchu (TW)

(21) Appl. No.: 19/308,498

(22) Filed: Aug. 25, 2025

(30) Foreign Application Priority Data
Apr. 2, 2025 (TW) 114112697

(51) Int. Cl. C07C 43/205; C08F 12/34; C08K 3/36

(52) U.S. Cl. CPC C07C 43/2055; C08F 12/34; C08K 3/36; C07C 2601/16; C08K 2201/005; C08K 2201/014

(57) ABSTRACT

A composition includes 3 to 90 parts by weight of a compound A and 10 to 97 parts by weight of a compound B, a compound C, or a combination thereof. When the composition includes the compound A and the compound B, the compound A and the compound B have a total weight of 100 parts by weight. The compound A has a chemical structure of

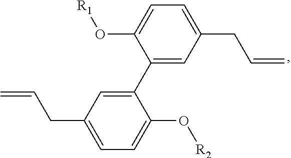

wherein each of R₁ and R₂ is independently of

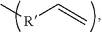

and R′ is

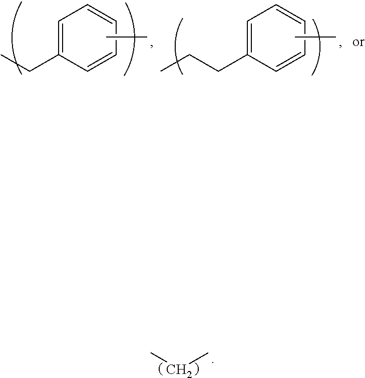

The compound B has one or two terminal double bonds, and the compound C has three to six terminal double bonds. The compound A and the compound C have different chemical structures.

### CROSS REFERENCE TO RELATED APPLICATIONS

The present application is based on, and claims priority from, Taiwan Application Serial Number 114112697, filed on Apr. 2, 2025, the disclosure of which is hereby incorporated by reference herein in its entirety.

### TECHNICAL FIELD

The technical field relates to compound, composition, and a package structure.

### BACKGROUND

As the era of 5G high-frequency approaches, the development of packaging technology will have to be altered as the specifications for wireless communication change. For example, the packaging material in AiP (Antenna in Package) must exhibit a low dissipation factor. Reducing the dielectric properties (Dk, Df) of packaging materials is the most important consideration in the reduction of the dissipation factor. As such, material structure design has gradually trended towards the use of hydrocarbon resin or polyolefin materials. Hydrocarbon resin or polyolefin materials often originate from petrochemical raw materials. Petrochemical raw materials are not sustainable materials. Therefore, it is imperative to develop bio-based resins to meet sustainable development.

### SUMMARY

One embodiment of the disclosure provides a compound, having a chemical structure of

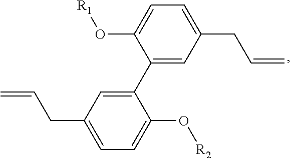

wherein each of R₁ and R₂ is independently of

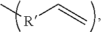

### and R′ is

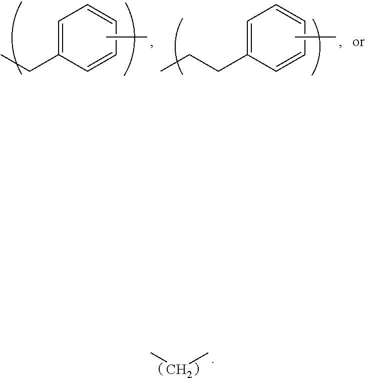

One embodiment of the disclosure provides a composition including 3 to 90 parts by weight of a compound A having a chemical structure of

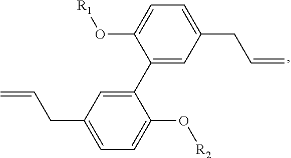

wherein each of R₁ and R₂ is independently of

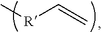

### and R′ is

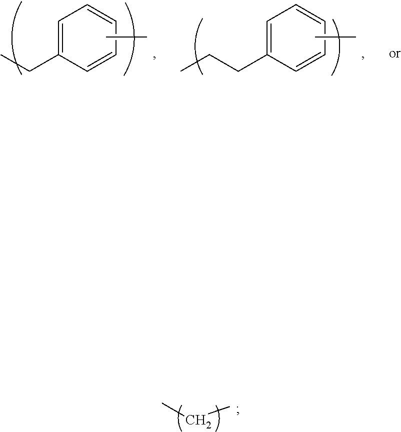

and 10 to 97 parts by weight of a compound B, a compound C, or a combination thereof. When the composition includes the compound A and the compound B, the total weight of the compound A and the compound B is 100 parts by weight. When the composition includes the compound A and the compound C, the total weight of the compound A and the compound C is 100 parts by weight. When the composition includes the compound A, the compound B, and the compound C, the total weight of the compound A, the compound B, and the compound C is 100 parts by weight. The compound B has one or two terminal double bonds, the compound C has three to six terminal double bonds, and the compound A and the compound C have different chemical structures.

One embodiment of the disclosure provides a package structure, including a substrate; a device disposed on the substrate; and a packaging material covering the substrate and the device, wherein the packaging material is formed by curing the described composition.

A detailed description is given in the following embodiments.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

One embodiment of the disclosure provides a compound, having a chemical structure of

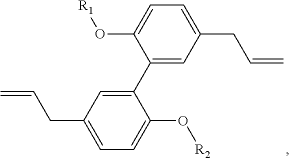

wherein each of R₁ and R₂ is independently of

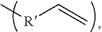

### and R′ is

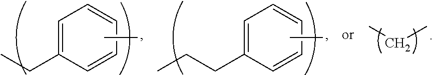

For example, magnolol, R₁-X, and R₂-X can be selected to perform a substitution reaction, and X can be halogen such as bromine, chlorine, or iodine. The magnolol can be a biomass material to increase the biomass content in products for meeting environmental protection requirements. The magnolol has a chemical structure of

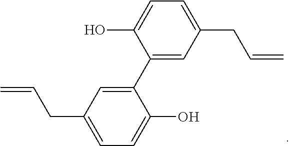 [SMILES: C=CCc1ccc(O)c(-c2cc(CC=C)ccc2O)c1]

This synthesis way is only for illustration, and one skilled in the art may adopt any path to synthesize the above compound and is not limited to the described path.

In some embodiments, the compound has a chemical structure of

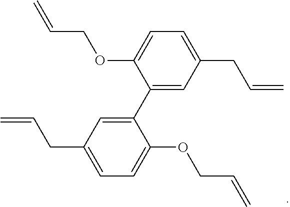 [SMILES: C=CCOc1ccc(CC=C)cc1-c1cc(CC=C)ccc1OCC=C]

In some embodiments, the compound has a chemical structure of

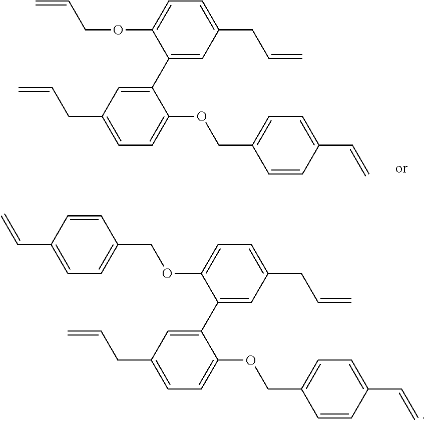

One embodiment of the disclosure provides a composition including 3 to 90 parts by weight of the described compound serving as a compound A, and 10 to 97 parts by weight of a compound B, a compound C, or a combination thereof. In some embodiments, when the composition includes the compound A and the compound B (without the compound C), the total weight of the compound A and the compound B is 100 parts by weight. In some embodiments, when the composition includes the compound A and the compound C (without the compound B), the total weight of the compound A and the compound C is 100 parts by weight. In some embodiments, when the composition includes the compound A, the compound B, and the compound C, the total weight of the compound A, the compound B, and the compound C is 100 parts by weight. In some embodiments, the compound B has one or two terminal double bonds, and the compound C has three to six terminal double bonds. The compound A and the compound C have different chemical structures. In some embodiments, the composition includes the compound A and the compound B without the compound C. If the amount of the compound A is too high, (e.g., the composition includes the compound A without the compound B, the compound C, or the combination thereof), the cured composition will be too hard and brittle. If the amount of the compound A is too low, the dissipation factor of the cured composition will be too high. In some embodiments, the composition can be used as packaging material.

In some embodiments, the compound B is a compound having a terminal styrene group. In some embodiments, the compound B is divinylbenzene, 4-tert-butylstyrene, 4-tert-butoxystyrene, or a combination thereof. In some embodiments, the compound C is triallyl isocyanurate, triallyl cyanurate, 2,2-bis[3-allyl-4-(4-vinylbenzyl)phenyl] propane, or a combination thereof.

In some embodiments, the composition further includes an inorganic powder, and the inorganic powder occupies 60% to 90% of the total weight of the composition (i.e., the inorganic powder filling rate is 60% to 90%) to further reduce the dissipation factor of the cured composition. In some embodiments, the inorganic powder can be silicon oxide, aluminum oxide, aluminum nitride, another suitable inorganic powder, or a combination thereof.

One embodiment of the disclosure provides a package structure, including a substrate; a device disposed on the substrate; and a packaging material covering the substrate and the device, wherein the packaging material is formed by curing the described composition. In some embodiments, the composition further includes an inorganic powder, and the inorganic powder occupies 60% to 90%. In some embodiments, the device is a chip. In some embodiments, the package structure may further include another device disposed on the packaging material (i.e., the packaging material is disposed between the device and the other device). In these embodiments, the device, the other device, and a bonding structure such as a bump between the device and the other device can be formed, and the composition is then filled into the gap between the device and the other device, and the composition is then cured to form the packaging material. The packaging material is so-called underfill material, potting material, or molding material. In addition, the composition can be applied onto the device by any method, and then cured to form the packaging material.

Below, exemplary embodiments will be described in detail so as to be easily realized by a person having ordinary knowledge in the art. The inventive concept may be embodied in various forms without being limited to the exemplary embodiments set forth herein. Descriptions of well-known parts are omitted for clarity.

### EXAMPLES

In following Examples, the viscosity of the composition was measured as follow: 1 g of the composition was placed into a viscometer (BROOLFIELD87333) to measure its viscosity at 25° C. and a rotation speed of 10 rpm.

In following Examples, the composition was cured as follow: 10 g of the composition was poured into a mold of 80 mm×15 mm×3 mm, repeatedly vacuum degassed, and placed in a high-temperature oven to be heated to 100° C. and maintained at 100° C. for 1 hour, and then heated to 135° C. and maintained at 135° C. for 6 hours.

In following Examples, the glass transition temperature (Tg) of the cured composition was measured as follow: the cured composition was cut and ground as a bulk material of 60 mm×15 mm×3 mm, and placed in Dynamic Mechanical Analyzer-Q800 (DMA) to be tested. The test was performed under nitrogen at a testing temperature of 0° C. to 300° C., and the heating rate was 10° C. per minute. The highest point of Tan Delta was Tg. The thermal decomposition temperature (Td) of the cured composition was measured as follow: the cured composition was heated to 650° C. in a thermogravimetric analyzer (TGA 550) by a heating rate of 10° C. per minute, and the temperature at which the weight loss being 95% was Td.

In following Examples, the dielectric constant, dissipation factor, and formability of the cured composition were measured as follow: the composition was poured into a mold composed of two glass plates (80 mm×80 mm) and a PTFE spacer having a thickness of 0.5 mm to be vacuum degassed, and then placed into a high-temperature oven to be heated to 100° C. and maintained at 100° C. for 1 hour, and then heated to 135° C. and maintained at 135° C. for 6 hours to obtain a cured composition. The dielectric constant (Dk) and dissipation factor (Df) of the cured composition were measured by a vector network analyzer with an open resonator at 25° C. under a frequency of 28 GHz and 50 GHz. The integrity of the dielectric sample after being cured was observed to confirm the formability of the cured composition. The formability of the cured composition was good when the sample was intact. The formability of the cured composition was common when the sample was slightly cracked. The formability of the cured composition was poor when the sample could not be shaped.

### Synthesis Example 1

10 g of magnolol (37.55 mmol) was dissolved in 200 mL of solvent DMSO. 5 g of NaOH was then added to the solution to be stirred at room temperature for 30 minutes. 12.73 g of 4-vinylbenzyl chloride (VBC) was then added to the solution to react at room temperature for 4 hours. Water (200 mL) was then added to the solution, which was then extracted by dichloromethane 3 times. The organic phase was collected and condensed to obtain a solid crude, which was washed by hexane and then dried to obtain white product (15 g). The hydrogen spectrum of the product was shown below: ¹H-NMR (500 MHZ, CDCl₃): δ3.00 (d, 4H), 4.95 (s, 4H), 5.00~5.18 (m, 4H), 5.22 (d, 2H), 5.73 (d, 2H), 6.01 (m, 2H), 6.60~6.75 (dd, 2H), 6.95 (b, 2H), 7.10~7.20 (m, 8H), 7.25~7.35 (m, 4H). The product had a chemical structure of

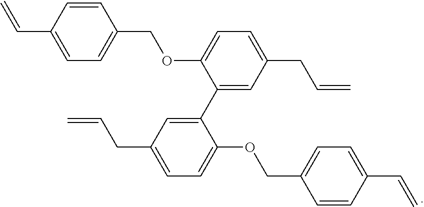 [SMILES: C=CCc1ccc(OCc2ccc(C=C)cc2)c(-c2cc(CC=C)ccc2OCc2ccc(C=C)cc2)c1]

### Synthesis Example 2

60 g of 2,2′-diallylbisphenol A and 550 mL of acetone were stirred and mixed. 15.6 g of NaOH was then added to the mixture to be stirred for 30 minutes. 72 g of VBC was then added to the mixture to react at room temperature overnight. The reaction result was filtered to remove solid thereof, and the filtrate was dried to obtain a solid crystal. The solid crystal was washed by methanol and then dried to obtain white product 2,2-Bis[3-allyl-4-(4-vinylbenzyl)phenyl] propane (AV-BPA). AV-BPA had a chemical structure of

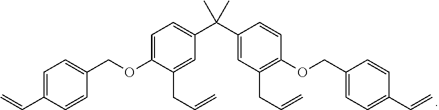 [SMILES: C=CCc1cc(C(C)(C)c2ccc(OCc3ccc(C=C)cc3)c(CC=C)c2)ccc1OCc1ccc(C=C)cc1]

### Example 1

50 parts by weight of the product in Synthesis Example 1 serving as a compound A, 50 parts by weight of a crosslinking agent 4-tert-butylstyrene (tBS) serving as a compound B, and 3 parts by weight of an initiator tert-butyl peroxy-3,5,5-trimethylhexanoate (TBPIN) were evenly mixed and stirred at room temperature to form a composition. The composition was cured to obtain a cured composition, which had Td of 376.5, a dielectric constant of 2.54 and a dissipation factor of 0.0075 under 28 GHz, and a dielectric constant of 2.51 and a dissipation factor of 0.0075 under 50 GHz. The formability of the cured composition was excellent.

### Example 2

25 parts by weight of the product in Synthesis Example 1 serving as a compound A, 50 parts by weight of tBS serving as a compound B, 25 parts by weight of AV-BPA serving as a compound C, and 3 parts by weight of the initiator TBPIN were evenly mixed and stirred at room temperature to form a composition. The composition was cured to obtain a cured composition, which had Td of 376.12, a dielectric constant of 2.51 and a dissipation factor of 0.0044 under 28 GHz, and a dielectric constant of 2.48 and a dissipation factor of 0.0043 under 50 GHz. The formability of the cured composition was excellent.

### Example 3

60 parts by weight of the product in Synthesis Example 1 serving as a compound A, 40 parts by weight of tBS serving as a compound B, and 3 parts by weight of the initiator TBPIN were evenly mixed and stirred at room temperature to form a composition. The composition was cured to obtain a cured composition, which had Td of 376.12, a dielectric constant of 2.56 and a dissipation factor of 0.0060 under 28 GHz, and a dielectric constant of 2.54 and a dissipation factor of 0.0055 under 50 GHz. The formability of the cured composition was excellent.

### Example 4

80 parts by weight of the product in Synthesis Example 1 serving as a compound A, 20 parts by weight of tBS serving as a compound B, and 3 parts by weight of the initiator TBPIN were evenly mixed and stirred at room temperature to form a composition. The composition was cured to obtain a cured composition, which had Td of 376.12, a dielectric constant of 2.64 and a dissipation factor of 0.0067 under 28 GHz, and a dielectric constant of 2.61 and a dissipation factor of 0.0065 under 50 GHz. The formability of the cured composition was excellent.

### Example 5

50 parts by weight of the product in Synthesis Example 1 serving as a compound A, 50 parts by weight of tBS serving as a compound B, 3 parts by weight of the initiator TBPIN (in which the compound A, the compound B, and TBPIN had a total weight of 15 g), 59.5 g of silicon dioxide powder FB-9454 (commercially available from Denka, having a size of 20 micrometers), 25.5 g of silicon dioxide powder FB-35 (commercially available from Denka, having a size of 12 micrometers), and 0.28 g of a dispersant BYK-145 (commercially available from BYK) were evenly mixed and stirred at room temperature to form a composition. The inorganic powder filling rate in the composition was 85%. The composition had a viscosity of 2878 cPa·s at 25° C. The composition was cured to obtain a cured composition, which had Tg of 192.0, a dielectric constant of 3.34 and a dissipation factor of 0.0015 under 28 GHz, and a dielectric constant of 3.31 and a dissipation factor of 0.0010 under 50 GHz. The formability of the cured composition was excellent.

### Example 6

25 parts by weight of the product in Synthesis Example 1 serving as a compound A, 50 parts by weight of tBS serving as a compound B, 25 parts by weight of AV-BPA serving as a compound C, 3 parts by weight of the initiator TBPIN (in which the compound A, the compound B, the compound C, and TBPIN had a total weight of 15 g), 59.5 g of silicon dioxide powder FB-9454, 25.5 g of silicon dioxide powder FB-35, and 0.28 g of the dispersant BYK-145 were evenly mixed and stirred at room temperature to form a composition. The inorganic powder filling rate in the composition was 85%. The composition had a viscosity of 5258 cPa·s at 25° C. The composition was cured to obtain a cured composition, which had Tg of 188.8, a dielectric constant of 3.35 and a dissipation factor of 0.0013 under 28 GHz, and a dielectric constant of 3.34 and a dissipation factor of 0.0012 under 50 GHz. The formability of the cured composition was excellent.

### Example 7

3.3 parts by weight of the product in Synthesis Example 1 serving as a compound A, 50 parts by weight of tBS serving as a compound B, 46.7 parts by weight of AV-BPA serving as a compound C, 3 parts by weight of the initiator TBPIN (in which the compound A, the compound B, the compound C, and TBPIN had a total weight of 17 g), 58.1 g of silicon dioxide powder FB-9454, 24.9 g of silicon dioxide powder FB-35, and 0.32 g of the dispersant BYK-145 were evenly mixed and stirred at room temperature to form a composition. The inorganic powder filling rate in the composition was 83%. The composition had a viscosity of 2080 cPa·s at 25° C. The composition was cured to obtain a cured composition, which had Tg of 189.4, a dielectric constant of 3.29 and a dissipation factor of 0.0016 under 28 GHz, and a dielectric constant of 3.26 and a dissipation factor of 0.0013 under 50 GHz. The formability of the cured composition was excellent.

### Comparative Example 1

50 parts by weight of tBS serving as a compound B, 50 parts by weight of AV-BPA serving as a compound C, 3 parts by weight of the initiator TBPIN (in which the compound B, the compound C, and TBPIN had a total weight of 17 g), 58.1 g of silicon dioxide powder FB-9454, 24.9 g of silicon dioxide powder FB-35, and 0.32 g of the dispersant BYK-145 were evenly mixed and stirred at room temperature to form a composition. The inorganic powder filling rate in the composition was 83%. The composition had a viscosity of 3192 cPa·s at 25° C. The composition was cured to obtain a cured composition, which had Tg of 190.9, a dielectric constant of 3.21 and a dissipation factor of 0.0034 under 28 GHz, and a dielectric constant of 3.19 and a dissipation factor of 0.0032 under 50 GHz. The formability of the cured composition was excellent.

### Comparative Example 2

6.5 g of epoxy resin CELLOXIDE 2021P (commercially available from Daicel), 3.5 g of bisphenol F epoxy resin EXA-830LVP (commercially available from DIC), 14.5 g of anhydride HN-2200 (commercially available from Hitachi Chemical Co., Ltd.), 0.5 g of vinyltriethoxy silane, 60 g of silicon dioxide powder FB-9454, and 15 g of silicon dioxide powder FB-35 were evenly mixed and stirred at room temperature to form a composition. The inorganic powder filling rate in the composition was 75%. The composition had a viscosity of 46500 cPa·s at 25° C. The composition was cured to obtain a cured composition, which had Tg of 175, and a dielectric constant of 3.51 and a dissipation factor of 0.011 under 28 GHz. The formability of the cured composition was excellent.

It will be apparent to those skilled in the art that various modifications and variations can be made to the disclosed methods and materials. It is intended that the specification and examples be considered as exemplary only, with the true scope of the disclosure being indicated by the following claims and their equivalents.

What is claimed is:

1. A compound, having a chemical structure of 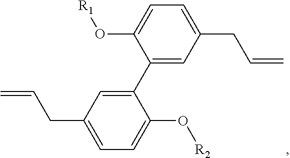
wherein each of R₁ and R₂ is independently of 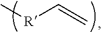
and R′ is

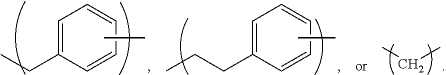

2. The compound as claimed in claim 1, having a chemical structure of

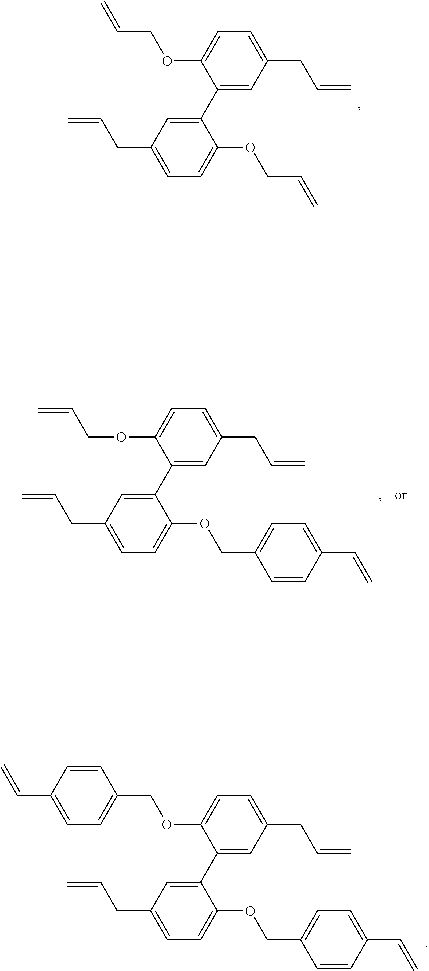

3. A composition, comprising:
3 to 90 parts by weight of a compound A having a chemical structure of 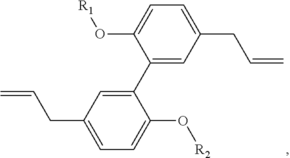
wherein each of R₁ and R₂ is independently of 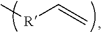
and R′ is 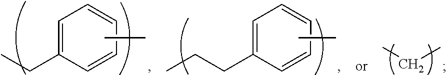
and
10 to 97 parts by weight of a compound B, a compound C, or a combination thereof,
when the composition includes the compound A and the compound B, a total weight of the compound A and the compound B is 100 parts by weight;
when the composition includes the compound A and the compound C, a total weight of the compound A and the compound C is 100 parts by weight;
when the composition includes the compound A, the compound B, and the compound C, a total weight of the compound A, the compound B, and the compound C is 100 parts by weight,
wherein the compound B has one or two terminal double bonds;
wherein the compound C has three to six terminal double bonds; and
wherein the compound A and the compound C have different chemical structures.

4. The composition as claimed in claim 3, wherein the compound B is a compound having a terminal styrene group.

5. The composition as claimed in claim 3, wherein the compound B is divinylbenzene, 4-tert-butylstyrene, 4-tert-butoxystyrene, or a combination thereof.

6. The composition as claimed in claim 3, wherein the compound C is triallyl isocyanurate, triallyl cyanurate, 2,2-bis[3-allyl-4-(4-vinylbenzyl)phenyl] propane, or a combination thereof.

7. The composition as claimed in claim 3, further comprising an inorganic powder, and the inorganic powder occupies 60% to 90% of a total weight of the composition.

8. The composition as claimed in claim 7, wherein the inorganic powder comprises silicon oxide, aluminum oxide, aluminum nitride, or a combination thereof.

9. A package structure, comprising:
a substrate;
a device disposed on the substrate; and
a packaging material covering the substrate and the device, wherein the packaging material is formed by curing the composition as claimed in claim 3.
